(19) United States
(12) Patent Application Publication
(10) Pub. No.: US 2012/0083751 A1
(43) Pub. Date: Apr. 5, 2012

(54) IMPLANTABLE DRUG DELIVERY DEPOT FOR SUBCUTANEOUS DELIVERY OF FLUIDS

(76) Inventor: Michael J. DALTON, Evanston, IL (US)

(21) Appl. No.: 13/324,358

(22) Filed: Dec. 13, 2011

Related U.S. Application Data
(62) Division of application No. 11/252,329, filed on Oct. 17, 2005.

(51) Int. Cl. A61M 5/00

(52) U.S. Cl. 604/288.01

(57) ABSTRACT

The port includes an elastomeric hollow port body having a first end and a second end, a first port end portion sealingly attached to the first end of the port body and a second port end portion attached to the second end of the port body. The second port end portion includes an outlet for fluid communication with a fluid delivery tube. The elastomeric hollow port body also includes an inner surface and an outer surface, the inner surface forming a lumen for receiving fluid.

### RELATED APPLICATIONS

This application claims priority to, and the benefit of, U.S. patent application. The present Ser. No. 11/252,329 filed on Oct. 17, 2005 by the present applicant. The entirety of that application is incorporated herein by reference.

### TECHNICAL FIELD

The technical field of this disclosure relates generally to medical devices, and more specifically to implantable drug delivery depots or ports for the subcutaneous delivery of fluids to the body.

### BACKGROUND OF THE INVENTION

There are many devices and methods for delivering fluids to a body. Implantable drug delivery depots, also known as ports, are just one example of a group of devices commonly used to deliver fluids to the body of laboratory animals as well as humans. These implantable ports are implanted between the skin and underlying fascia of the body and allow for the injection of fluids through a self-sealing septum or diaphragm located just under the skin and connected to a catheter or outlet tube which is placed in a vein. The implantable port is connected to a catheter or an outlet tube that is placed in a vein. Self-sealing septum have been used in the field of oncology for many years. The design of currently available implantable ports requires a flat silicone disk contained and compressed between two rigid frames. These frames with openings for the silicone compresses the silicone so that any puncture to the silicone is closed by excess material forcing the hole closed. The design, therefore, relies on a rigid top and bottom surface to compress the silicone in such a manner as to enable a puncture to close when the needle is removed. Significant limitations of this design is that the size of the septum opening is limited and that the plane of the septum must be flat.

Other designs of implantable ports utilize top and bottom wire screen or a wire matrix to compress the silicone. In these designs, openings within the wire matrix or screen allow the needle to pass through the silicone. One significant limitation of these wire mesh designs is that the openings must be large in order to provide a needle passage.

Another limitation of many of the implantable ports currently available is that they are bulky and have a large profile. These large profile ports are difficult to implant without making large incisions through the skin of the laboratory animal or human being. Furthermore, these large ports have limited applications due to the inability of the clinician to implant the port in small areas of the body or in small animals.

Still other ports are composed of several parts that must meet exacting standards, making the manufacture of the port both time consuming and expensive.

It would be desirable, therefore, to provide a device that overcomes these, and other, disadvantages.

### SUMMARY OF THE INVENTION

One embodiment of the invention provides an implantable port. The implantable port comprises an elastomeric hollow port body having a first end and a second end, a first port end portion sealingly attached to the first end of the port body and a second port end portion attached to the second end of the port body. The second port end portion includes an outlet for fluid communication with a fluid delivery tube. The elastomeric hollow port body also includes an inner surface and an outer surface, the inner surface forming a lumen for receiving fluid.

Another embodiment of the invention provides an implantable system for delivering fluid subcutaneously. The implantable system includes a port device having a port body, a first end and a second end. The port body, first end and second end form a lumen. The system further includes an elongate delivery tube attached to and in fluid communication with the port device.

Yet another embodiment provides a method of forming an implantable system for delivering fluid subcutaneously. The method comprises the steps of providing a hollow silicone tube having a uniform density and inverting the hollow silicone tube to form a port body having a silicone density gradient. The method further includes inserting a rigid support member into a lumen of the inverted silicone tube, attaching a first end cap and a second end cap to a first end and a second end of the inverted silicone tube and attaching a fluid delivery tube to the second end cap.

The present invention is illustrated by the accompanying drawings of various embodiments and the detailed description given below. The drawings should not be taken to limit the invention to the specific embodiments, but are for explanation and understanding. The drawings are not necessarily drawn to scale. The detailed description and drawings are merely illustrative of the invention rather than limiting, the scope of the invention being defined by the appended claims and equivalents thereof. The foregoing aspects and other attendant advantages of the present invention will become more readily appreciated by the detailed description taken in conjunction with the accompanying drawings.

### BRIEF DESCRIPTION OF THE FIGURES

FIG. 1 illustrates a perspective view of one embodiment of a system for subcutaneous delivery of fluids in accordance with the present invention;

FIG. 2 illustrates a partial cutaway side view of one embodiment of a port device for subcutaneous delivery of fluids in accordance with the present invention;

FIGS. 3A and 3B illustrate cross sections of silicone tubing utilized in the manufacture of one embodiment of a device for subcutaneous delivery of fluid in accordance with the present invention;

FIG. 4 illustrates a cross section of another embodiment of a port device for the subcutaneous delivery of fluids in accordance with the present invention; and

FIG. 5 illustrates a cross section of another embodiment of a port device for the subcutaneous delivery of fluids in accordance with the present invention.

FIG. 6 illustrates a rigid support member made in accordance with one embodiment of the present invention;

FIG. 7 illustrates a cross section of another embodiment of a port device for subcutaneous delivery of fluids in accordance with the present invention

FIGS. 7A and 7B illustrate a cross section of one embodiment of a port body of the port device illustrated in FIG. 7;

FIGS. 8A and 8B illustrate a cross section of another embodiment of a port body that may be utilized with the port device illustrated in FIG. 7;

FIG. 9 illustrate a cross section of another embodiment of a port body that may be utilized with the port device illustrated in FIG. 7;

FIG. 10 illustrates a perspective view of another embodiment of a system for subcutaneous delivery of fluids in accordance with the present invention; and

FIG. 11 is a flow chart of a method for forming one embodiment of a system for subcutaneous delivery of fluids in accordance with the present invention.

### DETAILED DESCRIPTION OF THE PRESENTLY PREFERRED EMBODIMENTS

Though the description is directed towards using the implantable system and port device in an animal such as, for example, a laboratory animal, those with skill in the art will recognize that the various embodiments of the implantable port herein described may be used in human beings and personal pets as well as any other animal under the care of medical personnel. In the below description, like reference numbers refer to like elements.

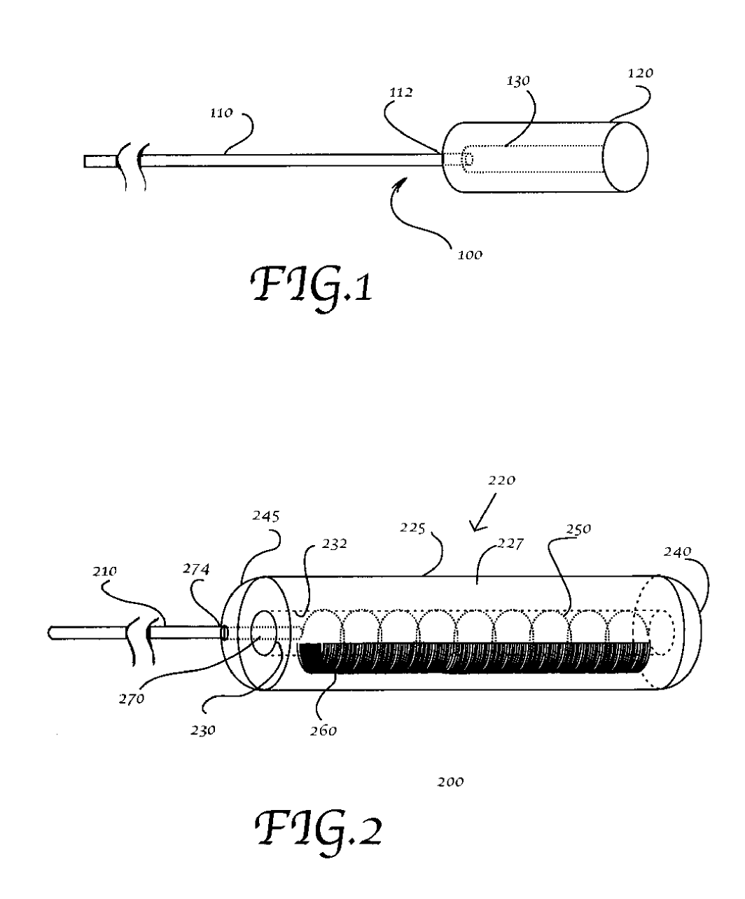

FIG. 1 illustrates a perspective view of an implantable system 100 for subcutaneous delivery of fluids in accordance with one aspect of the present invention. System 100 includes a delivery tube 110 and a port device 120. Delivery tube 110 is a hollow elongate tube operably attached to port device 120. Port device 120 includes a lumen 130 in fluid communication with delivery tube 110. In one embodiment, delivery tube 110 comprises a catheter. Delivery tube 110 may be positioned within the vasculature of the animal in any manner known to those with skill in the art. In another embodiment, delivery tube 110 is a cannula. In yet another embodiment, delivery tube 110 is any biomedically suitable delivery tube configured to deliver fluid to a delivery site. Delivery tube 110 may be positioned within the animal in any manner known to those with skill in the art.

In one embodiment, a first end 112 of delivery tube 110 may be fixedly attached to port device 120. In another embodiment, delivery tube 110 may be formed integrally with the port device 120. In other embodiments, the port device may include a connector for connecting the delivery tube 110 to the port body 120.

FIG. 2 illustrates a partial cutaway side view of one embodiment of a port device 220 for subcutaneous delivery of fluids of fluid delivery system 200, made in accordance with the present invention. Fluid delivery system 200 includes delivery tube 210 operably connected to port device 220. Delivery tube 210 may be implemented as described above for delivery tube 110.

Port device 220 comprises port body 225 and first and second end portions 240, 245, respectively. Port body 225 and end portions 240, 245 form lumen 230. Lumen 230 is in fluid communication with hollow delivery tube 210 via an opening 270 within second end 245. End portions 240, 245 are attached to port body 225 by adhesive or any other means known in the art that would provide a sealed lumen where the opening 270 provides the only exit for fluid injected into the lumen through port body 225 as will be discussed below. Port body 225 is composed of a biocompatible silicone elastomer. In one embodiment, port body 225 comprises a self-sealing silicone elastomer.

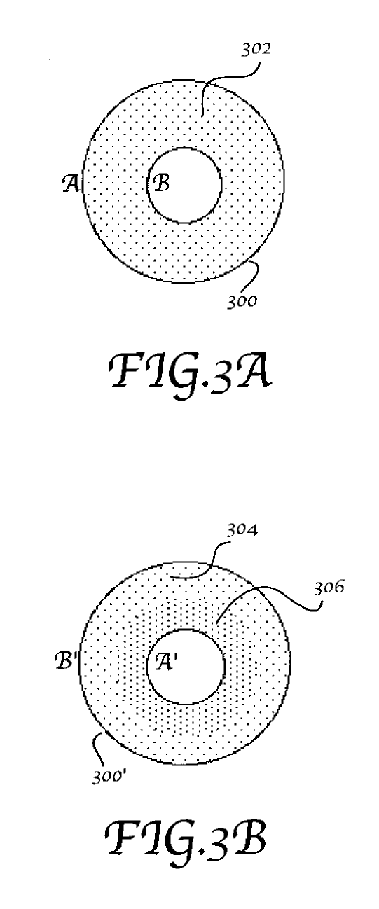

Referring to FIGS. 3A and 3B, FIGS. 3A and 3B illustrate representational cross sections of one embodiment of a silicone elastomer tube utilized in the formation of one embodiment of a self sealing port body 225. FIG. 3A illustrates a cross section of an elastomer tube 300 having an outer surface A and an inner surface B. As shown in FIG. 3A, in the relaxed states, elastomer tube 300 has a uniform density of silicone elastomer 302 between outer surface A and inner surface B. However, when elastomer tube 300 is turned in on itself it forms a stressed elastomer tube 300′ illustrated in FIG. 3B. Elastomer tube 300 is inverted in such a manner that the inner surface B becomes the outer surface B′ of the tube 300′ and the outer surface A becomes the inner surface A′. As a result of the inversion, the density of the silicone forms a radial gradient as illustrated in FIG. 3B. The radial gradient formed from the inversion of the elastomer tube is such that the density of the elastomer is greater nearest the inner surface A′ and lesser nearest the outer surface B′ as will be appreciated by one with skill in the art. In one example illustrated in FIG. 3B, the density of the elastomer is greater in the area 306 than in the area 304. In one embodiment, a cylindrical port device having a lumen with a longitudinal axis includes a port body comprised of a variable density silicone. In this embodiment, the density decreases radially from the longitudinal axis of the lumen.

The inversion of elastomer tube 300 into tube 300′ also provides a compressive force to the elastomer nearest inner surface A′. As the outer surface A becomes the inner surface A′, the material is compressed to take up the smaller space of the inner diameter of the tube 300′. The density gradient thus formed combined with the compressive force created by the inversion of the elastomer tube creates a self-sealing silicone tube that forms one embodiment of a port body such as, for example, port body 225 of FIG. 2.

Returning to FIG. 2, port body 225 is composed of a self-sealing silicone elastomer having a variable density as described above and illustrated in FIG. 3B. In one embodiment, the silicone elastomer tube is inverted as described above to form the port body 225 and ends 240, 245 are fixedly attached to the port body thereby forming lumen 230.

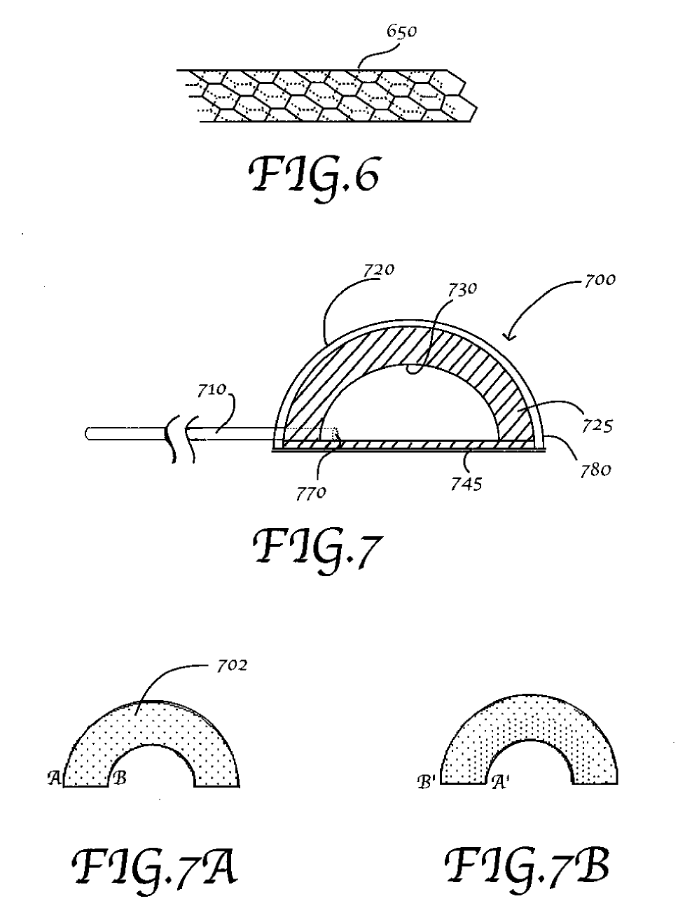

In another embodiment, a rigid support member 250 is positioned within lumen 230 prior to placement of ends 240, 245. In one embodiment, rigid support member 250 comprises a wire coil such as, for example, a compression spring. The wire coil is open or stretched to allow passage of a needle between the windings of the wire coil. In another embodiment, rigid support member 250 forms a framework that may be used to maintain the lumen in an open state. In this embodiment, rigid support member 250 has an inner diameter slightly larger than the diameter of the lumen created by the inversion of the elastomer tube. In this embodiment, the slightly larger diameter of the rigid support member 250 provides a compressive force to the inner surface of the elastomer tube that forms port body 225 by applying an outward force to the inner surface 232 of lumen 230. Those with skill in the art will recognize that rigid support member 250 may take other forms. FIG. 6 illustrates another embodiment of a rigid support member 650. Rigid support member 650 comprises a rigid open mesh structure. In one embodiment, rigid support member 650 comprises a stent, as are well known in the art. In another embodiment, rigid support member 650 is a mesh screen.

Port device 220 may also include a shield 260. Shield 260 may be composed of any puncture resistant metallic or polymeric base material. Shield 260 is positioned within port device 220 opposite to the needle puncture area. Shield 260 prevents the tip of the needle from completely passing through the port device 220 when in use. In effect, shield 260 provides a needle stop that indicates to the practitioner that the needle has been inserted properly and that the fluid may be delivered into the lumen 230 from the needle. In other embodiments, shield 260 may be positioned within the lumen 230 of port device 220, between layers of silicone elastomer in a multilayer embodiment, or on the outside of the port device. Shield 260 may be semicircular in shape as illustrated in FIG. 2. In other embodiments, shield 260 may be a flat elongate piece of material positioned within lumen 230 or between layers of silicone elastomer in a multilayer embodiment. In another embodiment, shield 260 is positioned between the surface of lumen 230 and rigid support member 250.

In one embodiment, port device 220 further includes an outer layer 227 that surrounds, at least, port body 225. In another embodiment, outer layer 227 encases the entire port device 220. Outer layer 227 comprises a silicone rubber material in an uncompressed or unstressed state. Outer layer 227 holds the stressed layer, or layers, of port device 220 together. Outer layer 227 may also prevent a cut or hole created by needle puncture from expanding as the stretched material attempts to spread the puncture. Outer layer 227 also provides a smooth outer surface of port device 220 to improve compatibility at the site of implantation. In other embodiments, lumen 230 is lined with a silicone rubber layer similar to or the same as outer layer 227.

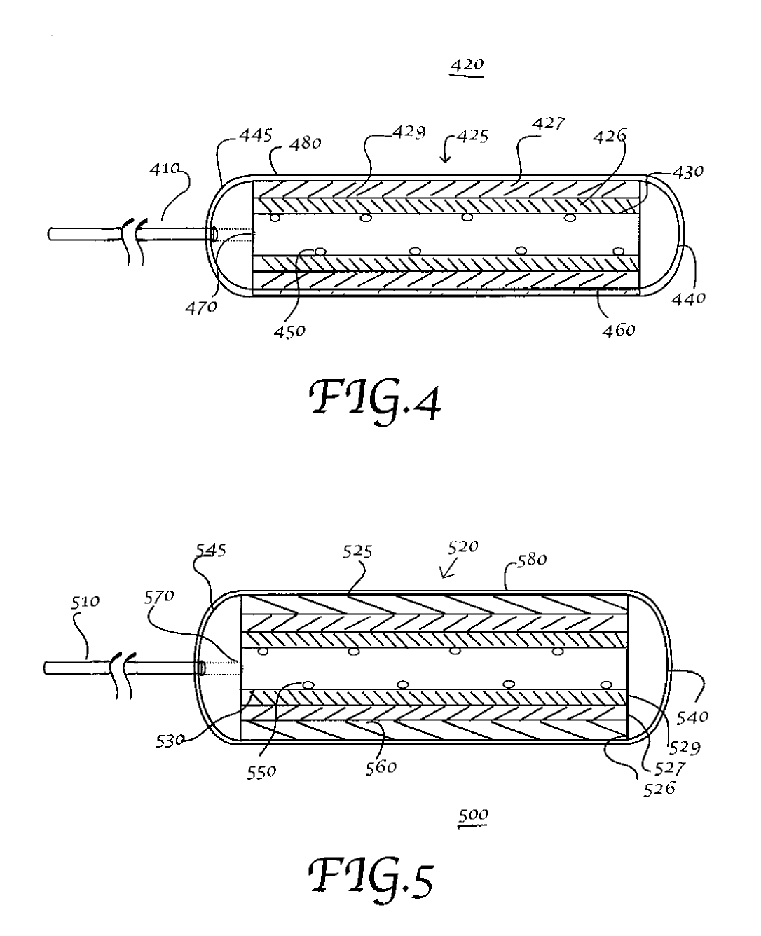

FIG. 4 illustrates a cross section of another embodiment of a fluid delivery system 400, made in accordance with the present invention. Fluid delivery system 400 includes delivery tube 410 operably connected to port device 420. Delivery tube 410 may be implemented as described above for delivery tube 110.

Port device 420 comprises port body 425 and first and second end portions 440, 445, respectively. Port body 425 and end portions 440, 445 form lumen 430. Lumen 430 is in fluid communication with hollow delivery tube 410 via an opening 470 within second end 445. End portions 440, 445 are attached to port body 425 by adhesive or any other means known in the art that would provide a sealed lumen where the opening 470 provides the only exit for fluid injected into the lumen through port body 425.

Port body 425 is a multilayer port body composed of a biocompatible silicone elastomer. In this embodiment, port body 425 is composed of a first elastomer tube 426 and a second elastomer tube 427. First elastomer tube 426 is inverted as discussed above creating a first layer of the self-sealing port body 425. Second elastomer tube 427 is also inverted in a similar manner as that for the first elastomer tube 426. In this embodiment, the inverted first elastomer tube 426 is positioned within a lumen 429 of inverted second elastomer tube 427. In one embodiment, the outside diameter of inverted first elastomer tube 426 is slightly larger than the diameter of lumen 429 so that, when assembled, an additional compressive force is created to increase the self-sealing ability of port device 420 when a needle is removed.

In the multilayer embodiment illustrated in FIG. 4, each layer has a similar gradient when inverted such that each layer is capable of self-sealing thereby providing a protective multiple redundancy for sealing a puncture.

Port device 420 also includes a rigid support member 450 that may be similar to or the same as that described for rigid support member 250 or 650 described above.

Port device 420 also includes a shield 460. In this embodiment, shield 460 is positioned on the outer surface of port device 420. Shield 460 may be attached by adhesive or any other means known to those with skill in the art. Shield 460 may be shaped as described above with regards to shield 260. In one embodiment the shape of shield 460 corresponds to the shape of the outer surface of the port device. In other embodiments composed of two inverted tubes, or layers, shield 460 may be placed between the layers.

Port device 420 also includes an outer layer 480. Outer layer 480 may be the same as, or similar to, outer layer 227 described above. Outer layer 480 encases port body 425 and shield 460.

Those with skill in the art will recognize that the port device may be composed of more than two layers. In embodiments that include multiple layers of silicone, shields may be placed between any of the layers or within the lumen as described above. In other embodiments, a wire coil or other rigid support member may be placed between adjacent layers of the elastomer tubes. In still other embodiments, more than one rigid support member may be placed in the port device. For example, in a three layer port body, a wire coil may be placed between the first and second layers and the second and third layers. The use of additional rigid support members may increase the compression of the silicone elastomer and provide an increased ability to seal a puncture when a needle is removed.

FIG. 5 illustrates a cross section of another embodiment of a fluid delivery system 500, made in accordance with the present invention. Fluid delivery system 500 includes delivery tube 510 operably connected to port device 520. Delivery tube 510 may be implemented as described above for delivery tube 110.

Port device 520 comprises port body 525 and first and second end portions 540, 545, respectively. Port body 525 and end portions 540, 545 form lumen 530. Lumen 530 is in fluid communication with hollow delivery tube 510 via an opening 570 within second end 545. End portions 540, 545 are attached to port body 525 by adhesive or any other means known in the art that would provide a sealed lumen where the opening 570 provides the only exit for fluid injected into the lumen through port body 525.

Port body 525 is a multilayer port body composed of a biocompatible silicone elastomer. In this embodiment, port body 525 is composed of a first elastomer layer 526 having a first density, a second elastomer layer 527 having a second density and third elastomer layer 529 having a third density. In one embodiment as illustrated in FIG. 5, the density of the layers increases from the outer most layer to the inner most layer. In one embodiment, the port body 525 is a single tube composed of multiple layers of silicone material having graduated densities. In this embodiment, the material increases in density from the outer most layer to the inner most layer, whereby the inner most layer has a substantially higher density of silicone than the outer layer.

In another embodiment, port body 525 is composed of a plurality of concentrically arranged elastomeric tubes. In this embodiment, a first tube having a first density is placed within the lumen of a second tube having a second density, the first tube having a greater density than the second tube. The second tube may then be placed within the lumen of a third tube having a lesser density than the second tube. In these multi-tube embodiments, the outer diameter of a tube is greater than the diameter of the lumen into which it will be inserted, thereby providing a compression force to the tube that is inserted into the lumen. The compression of one layer by an adjoining layer provides a self-sealing port body 525. In this embodiment, each layer is self-sealing, thereby providing multiple redundancy for sealing a needle puncture.

Port device 520 includes a rigid support member 550 disposed within lumen 530 the same as or similar to rigid support members 250 and 650 described above. Rigid support member 550 may comprise a compression spring that provides a compressive force to the inner layers of material to aid in sealing a puncture when a needle is removed.

Port device 520 may also include shield 560. Shield 560 is disposed between first layer 525 and second layer 527. Shield 560 may be the same or similar to the shields described above. Port device 520 may also include an outer layer 580. Outer layer 580 may be the same as, or similar to, outer layer 227 and 480 described above.

In other embodiments of the port device, the outer surface of the port device may be covered with a fabric layer to improve biocompatibility of the implanted device. In one embodiment, the covering comprises a Dacron® fiber material. In still other embodiments, the port device may include a coating of a therapeutic agent to prevent the formation of blood clots or to prevent tissue ingrowth.

FIG. 7 illustrates a cross section of another embodiment of a fluid delivery system 700 for subcutaneous delivery of fluids, made in accordance with the present invention. Fluid delivery system 700 includes delivery tube 710 operably connected to port device 720. Delivery tube 710 may be implemented as described above for delivery tube 110.

Port device 720 comprises port body 725 and port base 745. Port body 725 and port base 745 form lumen 730. Lumen 730 is in fluid communication with hollow delivery tube 710 via an opening 770 defined within port body 725. Port base 745 is attached to port body 725 by adhesive or any other means known in the art that would provide a sealed lumen where the opening 770 provides the only exit for fluid injected into the lumen through port body 725. Port base 745 may is composed of a rigid biocompatible material. In one embodiment, port base 745 acts as a shield to prevent the needle from exiting lumen 730. Port base may be composed of any suitable biocompatible metallic or polymer as are known in the art. Port body 725 is composed of a biocompatible silicone elastomer. Port device 720 may also include an outer layer 780 similar to, or the same as outer layers 480 and 580, described above.

Port body 725 is a dome-shaped structure comprising a self-sealing silicone elastomer having a density gradient similar to that described above. FIGS. 7A and 7B illustrate cross sections of a single layer elastomeric dome used in the construction of port device 720. FIG. 7A illustrates a cross section of the silicone material of port body 725 as it would appear in the relaxed state, having a uniform density from the outside surface A to the inside surface B. FIG. 7B illustrates a cross section of the silicone material as it would appear in the inverted stressed state, having a density gradient that increases from the outside surface B′ to the inside surface A′. During manufacture, the inverted stressed silicone dome is adhered to base 745 to form the self-sealing port body 725 of port device 720.

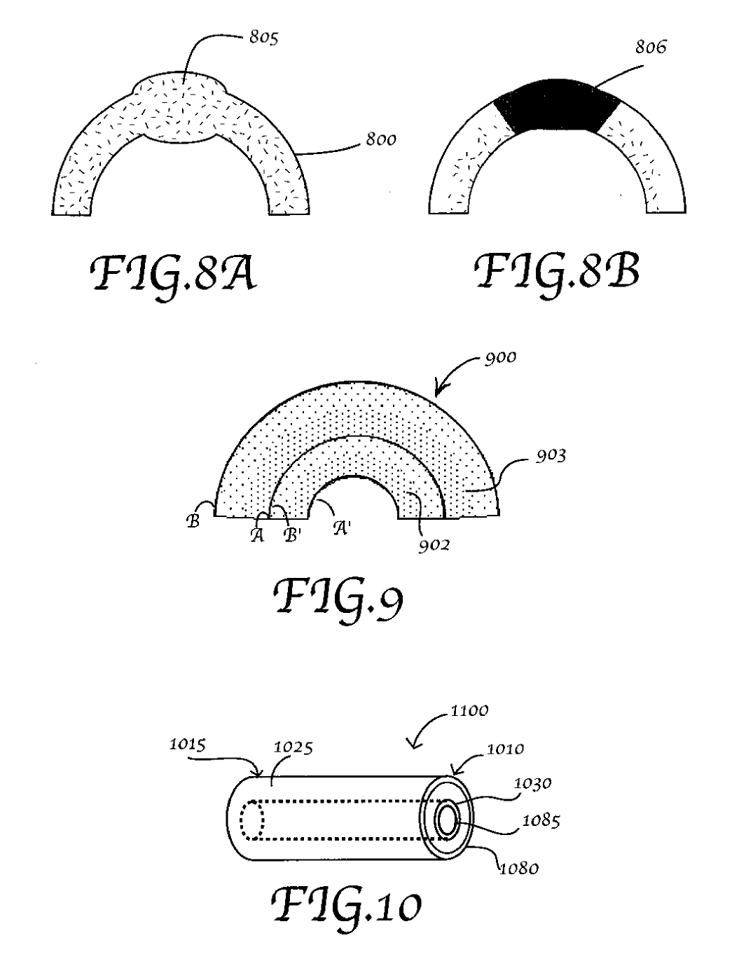

FIGS. 8A and 8B illustrate cross sections of another embodiment of an elastomeric dome used in the construction of port device 720. FIG. 8A illustrates a cross section of the silicone material 800 as it would appear in the relaxed state. In this embodiment, the silicone dome includes an area of increased thickness 805. FIG. 8B illustrates a cross section of the silicone material as it would appear in the inverted stressed state. As is apparent from these illustrations, the inversion of dome 800 creates an area of higher density 806 at the top of the dome. Furthermore, the inversion of this area of increased density 806 also provides a compressive force to the silicone material, thereby increasing the self-sealing capability of the port device.

FIG. 9 illustrates a cross section of another embodiment of an elastomeric dome 900 used in the construction of a dome-shaped port device 720 illustrated in FIG. 7. Elastomeric dome 900 comprises a multilayer dome composed of two inverted silicone domes 902, 903 similar to, or the same as those described in FIGS. 7A to 7B, above. In the inverted stressed state illustrated in FIG. 9, the multiple layered dome 900 includes two layers each having a density gradient that increases from the outside surface B to the inside surface A of each layer. Each inverted and stressed layer comprises a self-sealing layer of silicone that provides multiple redundancy for sealing a needle puncture.

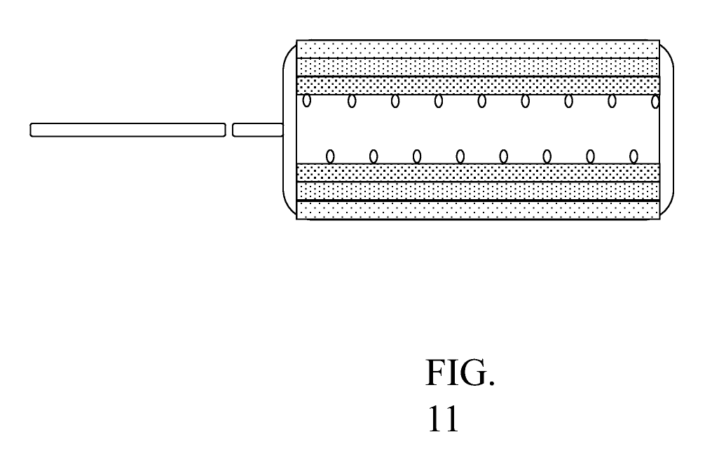

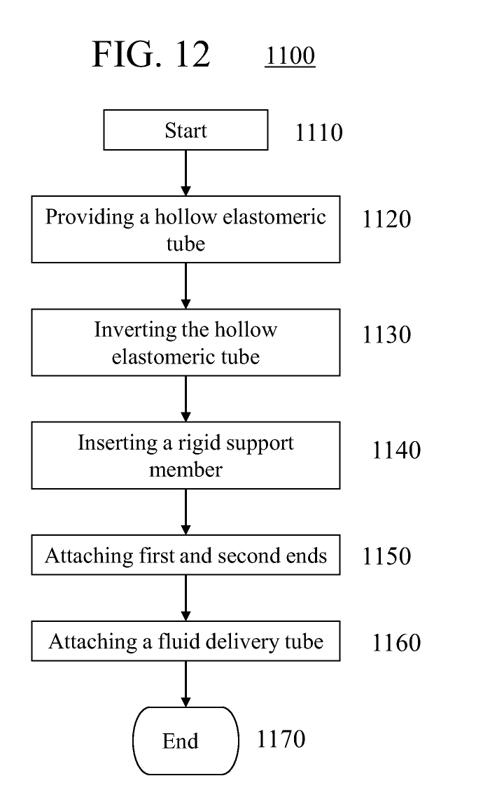

FIG. 10 illustrates another embodiment of a self-sealing port device 1000, made in accordance with the present invention. Port device 1000 comprises a cylindrically shaped port body 1025 having a lumen 1030 extending therethrough. Port device 1025 has an open first end 1010 and an open second end 1015. Port body 1025 is composed of a self-sealing silicone elastomeric material having a density gradient, as described above. Port body 1025 may be composed of single or multiple layers of inverted stressed silicone elastomer similar to, or the same as, the silicone port bodies described above in relation to FIGS. 1 to 5. Port device 1000 may also include an outer layer 1080 for encasing port body 1025. Outer layer 1080 is an uncompressed and unstressed layer of material as described above. In one embodiment, lumen 1030 is covered by an inner layer 1085 of the same or similar material as that of outer layer 1080. Layers 1080 and 1085 provide a smooth surface for blood compatibility.

Port device 1000 may be implanted in a vessel or other elongated structure in the body where multiple sites of injection are contemplated throughout a treatment or experimental procedure. Port device 1000 may be sized to have an outer diameter sufficiently larger than that of the diameter of the lumen of the vessel into which it is implanted in order to prevent migration of the device 1000 after implantation. In another embodiment, a rigid support member may be embedded within the silicone layer or between layers in a multiple layer embodiment to increase the compression of the material, as described above.

FIG. 11 is a flow chart of a method 1100 for forming one embodiment of a system for subcutaneous delivery of fluids. Method 1100 begins at 1110. An elastomeric hollow tube is provided for forming the hollow port body (Block 1120). The elastomeric hollow tube may be any one of those described above and illustrated in FIGS. 1 to 5. In one embodiment, the hollow tube is a silicone tube having a uniform density. The hollow tube is then inverted to create the density gradient (Block 1130). A rigid support member is inserted into the lumen of the inverted elastomeric tube to provide a compression force to the inner layer of the inverted tube (Block 1140). The rigid support member may be, for example, a spring, a stent or a rigid mesh, as described above. Once the rigid support member is inserted, the first and second ends may be attached, thereby forming a lumen for receiving fluid (Block 1150). One of the ends that are attached includes an opening for fluid communication with a delivery tube. Finally, a fluid delivery tube is attached to the end having the opening (Block 1160). The method of forming a system for subcutaneous delivery of fluids ends at 1170.

Variations and alterations in the design, manufacture and use of the system and method may be apparent to one skilled in the art, and may be made without departing from the spirit and scope of the present invention. While the embodiments of the invention disclosed herein are presently considered to be preferred, various changes and modifications can be made without departing from the spirit and scope of the invention. The scope of the invention is indicated in the appended claims, and all changes that come within the meaning and range of equivalents are intended to be embraced therein.

What is claimed is:

1. An implantable port, comprising:
an elastomeric hollow port body,
wherein the elastomeric hollow port body includes an inner surface and an outer surface, the inner surface forming a lumen for receiving fluid;
wherein the elastomeric hollow body comprises a dome portion and a base portion, the base portion sealingly attached to the dome portion, the dome portion and the base portion forming the lumen for receiving fluid, wherein the dome portion includes an opening for fluid communication with a fluid delivery device.

2. The device of claim 1 wherein the dome portion comprises an inverted silicone dome having a self-sealing density gradient.

3. The device of claim 2 wherein the inverted silicone dome comprises at least one layer of silicone material, each layer having a density gradient.
